(19) United States
(12) Patent Application Publication
(10) Pub. No.: US 2026/0090846 A1
(43) Pub. Date: Apr. 2, 2026

(54) A ROBOT FOR CATHETER OPERATION WITH FORCE MEASUREMENT

(71) Applicant: Shenzhen Tonglu Technology Co., LTD, Shenzhen (CN)

(72) Inventors: Yunfei Cao, Shenzhen (CN); Dwight Meglan, Needham, MA (US); Ann Xin, Lincoln, MA (US)

(73) Assignee: Shenzhen Tonglu Technology Co., LTD, Shenzhen (CN)

(21) Appl. No.: 19/110,093

(22) PCT Filed: Sep. 11, 2023
(86) PCT No.: PCT/CN2023/118088

§ 371 Date: Mar. 10, 2025

(30) Foreign Application Priority Data
Sep. 9, 2022 (CN) CN202211105526.7
Sep. 22, 2022 (CN) CN202211146397.6

(51) Int. Cl. A61B 34/30; A61B 90/00

(52) U.S. Cl. CPC A61B 34/30; A61B 90/06; A61B 2034/301; A61B 2034/305; A61B 2090/064

(57) ABSTRACT

Herein disclosed is a robot apparatus having two or more robot units, each robot unit is configured to maneuver on an intracavity surgical device, such as a catheter, a guide wire, or tools for laser ablation. Also included is a reciprocating conveyor having a motion converter, such as a scotch yoke, coupled with a rail slider to produce linear reciprocating movements to bring the robot units back and forth from and towards a patient. A load measuring device is also included to sense both the axial force and the torque exerted on the surgical device and based on the real time load measurement, to control the scotch yoke and the robot unit more accurately for inserting the intracavity surgical device into or withdrawing it from the surgical subject.

### CROSS-REFERENCE TO RELATED APPLICATIONS

This application claims priorities to and benefits of Chinese Patent Application Ser. No. 202211105526.7, entitled “A SURGICAL ROBOT”, filed on Sep. 9, 2022, and Chinese Patent Application Ser. No. 202211146397.6, entitled “A SURGICAL ROBOT WITH FORCE MEASUREMENT”, filed on Sep. 20, 2022, each of which is incorporated herein by reference in its entirety and for all purposes.

### TECHNICAL FIELD

This application relates generally to medical robot apparatus, particularly to a robot apparatus facilitating robot catheter manipulation on a surgical subject.

### BACKGROUND

Currently, medical equipment and robot-assisted surgery technologies are developing rapidly. The market has seen technologies involving surgical robot systems for laparoscopy, orthopedics, and vascular intervention entering trials, clinical trials or being launched into the market. However, a robot catheter operation without the force exerted on the catheter accurately measured in real time is dangerous to the surgical subject, as perforation or other kind incidences may happen. Existing robot devices lack the versatility and accuracy provided to the operations. Therefore, there is an urgent need to provide a robot apparatus that facilitate the nimble operations of a catheter safely and efficiently, sensing the force exerted onto the catheter in real time during the operation.

### SUMMARY

In accordance with a first aspect of the present disclosure, there is set forth is a robot apparatus that includes two or more robot units, each robot unit is configured to maneuver on an intracavity surgical device, such as a catheter, a guide wire, or tools for laser ablation. Also included is reciprocating conveyor having a motion converter, such as a scotch yoke, coupled with a rail slider to produce linear reciprocating moves to bring the robot units back and forth from and towards a patient. A load measuring device is also included to sense both the axial force and torque exerted on the surgical device and based on the real time load measurement, an operator can control the scotch yoke and the robot unit more accurately for inserting the intracavity surgical device into or withdrawing it from the surgical subject.

In accordance with yet another aspect of the present disclosure, there is set forth the herein disclosed the two or more robot units working with each other in a coordinated manner. For example, with one robot unit making a linear reciprocating movement while holding the catheter, the other robot unit is configured to let loose of the catheter. The roles reverse between the two robot units in the next cycle such that the catheter is inserted into or withdrawn from the surgical subject. The coordination of these grasp-move-release actions between the two robot units can be programmed such that the tool moves continuously even as the individual robot units have discontinuous movement.

In summary, the surgical robot with load measurement according to the present disclosure is intended not only to provide the operation of insertion or withdrawing a guidewire, catheter, angioplasty balloon or other endovascular tool, but also to achieve the task with more accurate real time measurement of the load exerted on the tool, increasing safety to the procedure and improving operation efficiency.

Accordingly, herein disclosed methods and apparatus are directed to solve one or more problems set forth above.

### BRIEF DESCRIPTION OF THE DRAWINGS

In the drawings, each reference numeral generally refers to the same part throughout the different views. Also, the drawings are not necessarily to be in scale, with emphasis instead generally being placed upon illustrating the principles of the disclosed systems and methods and are not intended as limiting. For clarity, not every component may be labeled in every drawing. In the following description, various embodiments are described with reference to the following drawings.

FIG. 1 is an exploded schematic view of an exemplary embodiment of a catheter robot assembly for operating catheters or the like in accordance with the present disclosure.

FIG. 2 is a perspective view of an exemplary embodiment of a conveyor assembly of the catheter robot assembly in accordance with the present disclosure.

FIG. 3 is another perspective view of an exemplary embodiment of a conveyor assembly of the catheter robot assembly in accordance with the present disclosure.

FIG. 4 is a prospective view of an exemplary embodiment of a load measuring assembly of the catheter robot assembly in accordance with the present disclosure.

FIG. 5 is another prospective view of the exemplary embodiment of the load measuring assembly of the catheter robot assembly in accordance with the present disclosure.

### DETAILED DESCRIPTION

The forgoing description of the partially reusable robot apparatus can be used for insertion or removal of catheters, as an example. It should be appreciated that the scope and spirit of this disclosure is not limited to this example.

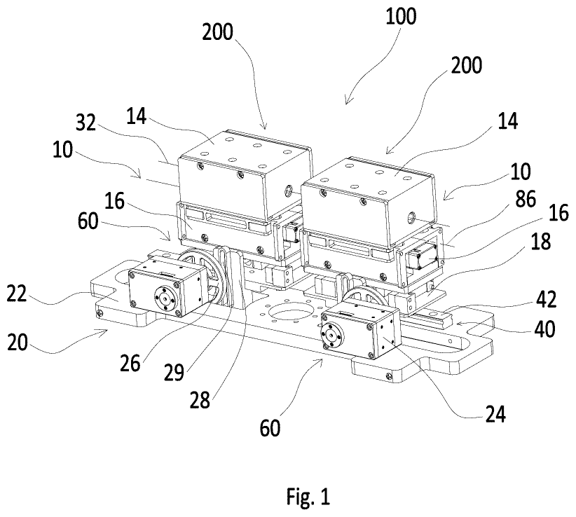

FIG. 1 is an exploded schematic view of an exemplary embodiment of a catheter robot assembly for operating catheters or the like in accordance with the present disclosure. For the clarity of the overall operational environment of the robot apparatus, some elements, although not shown in the drawings, are important to the present disclosure. These include a surgical subject to the far right hand side of FIG. 1, and a sterile barrier enveloping the “sterile side”, shown as a line sterile barrier 86, representing a plane. The directions of moving to or away from the surgical subject are called “back and forth”, or linear direction.

The forgoing description of the present disclosure is described in detail in conjunction with the accompanying drawings and embodiments. It should be understood that the specific embodiments herein described are only used to explain the present disclosure, not to limit the present disclosure.

In the present disclosure, unless otherwise clearly specified and limited, the terms “installation”, “attachment”, and “connection” should be understood in a broad sense, for example, it may be a fixed attachment, a detachable connection, or an integral body. It may also be an attachment that can move relatively. It may also be a mechanical connection or an electrical connection. It may be a direct or indirect connection through an intermediary. It may be an internal communication of two components or the interaction relationship between two components. The specific meanings of the above terms used in the present disclosure are to be understood by those skilled in the art according to specific situations.

In the description of the present disclosure, the terms “upper”, “lower”, “front”, “rear”, “left”, “right”, “horizontal”, “bottom”, “inner”, etc. indicate orientation or positional relationship in reference to the corresponding drawings. It is only for the convenience of describing the present disclosure, rather than indicating or implying that the referred device or component must have a specific orientation or must be constructed and operated in a specific orientation, therefore shall not be construed as a limitation of the disclosure.

In addition, the terms “first”, “second”, etc. are used for descriptive purposes only, and should not be understood as indicating or implying relative importance or implicitly specifying the quantity of the indicated technical features. Thus, a feature defined as “first”, “second”, etc. may expressly or implicitly include one or more of that kind of features.

The catheter robot assembly of the present disclosure can be used for delivery or withdrawal of intraluminal surgical devices, such as catheters. It should be understood that the term “intraluminal” includes lumens such as natural lumens, pan vascular lumens, and organ lumens. The term “intraluminal surgical device” may refer to any shape or any kind of catheter, guide wire, guide catheter, angioplasty catheter, or endoscope, various laparoscopes, tube mirrors, etc., including those suitable for vascular intervention, any catheter, guide wire, or consumable device for electrophysiology, structural heart disease and other procedures. The guide wires herein include but not limited to guide wires, loach guide wires, contrast guide wires and micro guide wires, etc. for guiding and supporting intracavitary surgical devices, and catheters include but not limited to guide catheters, micro catheters, contrast catheters, intermediate tubes (also known as intermediate catheters), thrombolytic catheters, balloon dilation catheters and ball dilation stent catheters and other diagnostic and therapeutic intracavitary surgical devices.

The terms “far” and “front” used in the present disclosure are directionally toward the surgical subject. The term “near” is the directionally away from the subject or to be near the surgical robot. The term “insert” refers to the direction in which the endoluminal surgical device is placed into the surgical subject. The term “retract” refers to the direction in which the endoluminal surgical device is withdrawn out of the subject.

It should further be noted that, when it is feasible, the embodiments of the present disclosure and various features in the embodiments may be combined with each other, and such combinations are all within the protection scope of the present disclosure.

Referring to FIG. 1, an exploded schematic diagram of an exemplary embodiment of a catheter robot assembly for operating catheters or the like is herein described.

As shown in FIG. 1, a catheter robot assembly 100 is configured to deliver or withdraw an intracavity surgical device, such as a catheter 32, being operated on surgical subject. Although the surgical subject is not shown, the subject is located further to the far right in the figure. For the clear presentation of the catheter robot assembly 100 of the present invention, although some components are omitted from the figure, they are still important.

In an exemplary embodiment, catheter robot assembly 100 of the present invention includes two catheter robot units 200. Each catheter robot unit 200 includes a catheter operation assembly 10, a reciprocating conveyor 20 and a load measuring assembly 30 located between the catheter operation assembly 10. Catheter operation assembly 10 includes an actuator 14 and a driving assembly 16. Actuator 14 is controlled by the driving assembly 16 in a non-contact manner. Driving assembly 16 is carried by a mounting plate 18, which is driven by the respective reciprocating conveyor 20 on a rail slider 40.

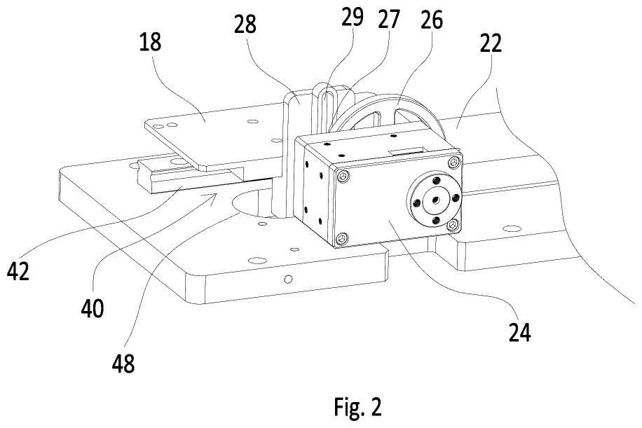

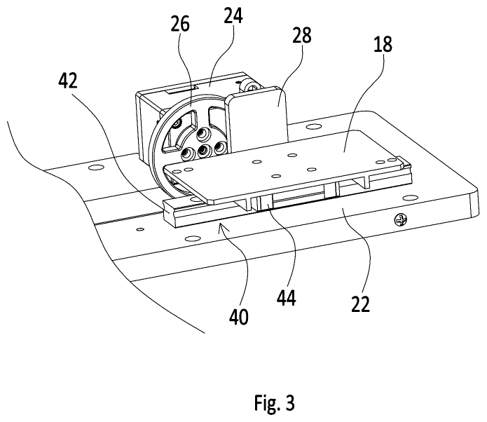

Referring to FIGS. 1-3, in this exemplary embodiment, reciprocating conveyor 20 includes a motor 24, a scotch yoke 60, and a piston linkage 28. Scotch yoke 60 includes a scotch disk 26, a disk pin 27 and a sliding yoke 29 attached to the piston linkage 28. Scotch yoke motor 24 is attached to base plate 22, which is placed substantially horizontal during operation. As shown in FIGS. 2 and 3, motor 24, being mounted on the base plate 22, and scotch yoke 60 are configured to work cooperatively as the reciprocating conveyer 20 to bring catheter robot units 200 forth and back, towards the surgical subject and away from the surgical subject. As such, scotch disk 26 is mounted rotatably onto an output shaft (not shown) of motor 24. The disk pin 27 of scotch disk 26 is configured to slidably fit into sliding yoke 29, which is positioned orthogonally to the base plate 22 and fixedly attached to a mounting plate 18. Respective piston linkage 28 is attached to the respective sliding yoke 29. The mounting plate 18 be seen in FIGS. 1 and 2, yoke motor 24 is fixedly attached to base plate 22. Yoke motor 24 is slidably fixed to the base plate 22 via rail slider 40, which is placed in a linear direction towards or away from the surgical subject.

It should be noted that scotch disk does not have to be a disk. It alternatively may be a rotating object carrying the disk pin 27. All of such alternation is within the scope of the present disclosure.

As can 24 and scotch disk 26 do not move linearly forth and back (towards and from the surgical subject) relevant to base plate 22. However, sliding yoke 29 and piston linkage 28 travel linearly forth and back relative to base plate 22. Slots 48 on base plate 22 are configured to accommodate sliding yoke 29 and piston linkage 28 and give space for their above described linear movements.

Referring to FIGS. 1-3, rail slider 40 functions as a piston of scotch yoke 60, with mounting plate 18 fixedly attached to sliding yoke 29 via piston linkage 28. Rail slider 40 includes a sliding rail 42 attached to the base plate 22 and a slide block 44 slidably fitted on sliding rail 42. Alternatively, piston linkage 28 of scotch yoke 60 is attached to slide block 44. When yoke motor 24 drives and rotates scotch disk 26, disk pin 27 on the disk 26 moves circularly in a vertical plane perpendicular to base plate 22, and it is configured to slide inside the sliding yoke and push the sliding yoke to move linearly towards and away from the surgical subject, facilitating the linear delivery or withdrawal of the intracavity surgical device 32, such as a catheter 32. For details, refer to a surgical robot described in Chinese patent application 202211105526.7, the entire content of which is incorporated this invention.

Still referring to FIGS. 1-3, as can be seen from the above description, driving assembly 16 is mounted on mounting plate 18 which is slidably fixed to the base plate 22 via rail slider 40, which is set in a linear motion by scotch yoke 60, in a direction towards or away from the surgical subject. Actuator 14 which is mounted on mounting plate 18 therefore subsequently moves linearly towards and away from the surgical subject. The resulting movement of surgical device, such as catheter 32 results from the compounded operation of actuator 14, with or without fixing clip 12 (not shown) alternately clamping the intracavity surgical device 32, and the movement of mounting plate 18. The intraluminal surgical device 32 is intermittently delivered into or withdrawn from the subject. And through the rotation of the reverse thrust motor 24, the linear motion distance and the real time position of the mounting plate 18 can be accurately known, thereby obtaining the motion distance and the real time position of the driving assembly 16 installed on the mounting plate 18 and actuator 14 which is linearly moved synchronously with mounting plate 18. As such, precision control of the intracavity surgical device 32, such as the catheter, can be achieved.

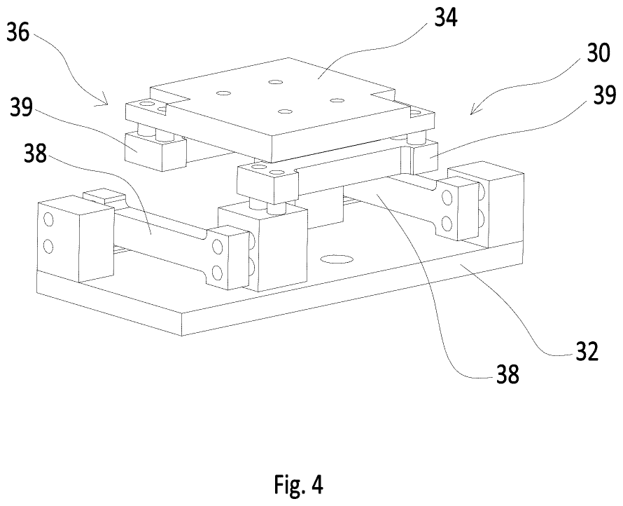

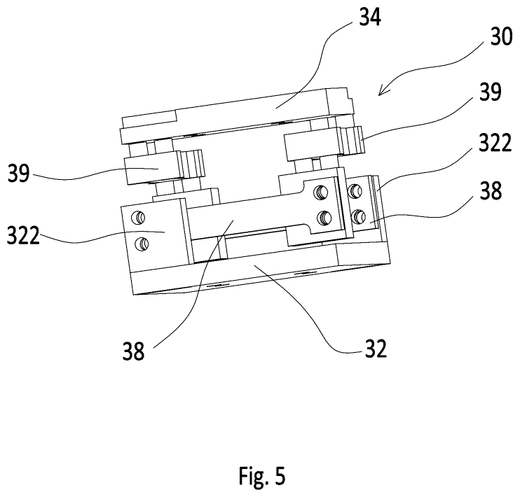

Reference is now made to FIGS. 4 and 5, the load measuring assembly 30 includes mounting plate 18 to be attached slide block 44, a top plate 34 for carrying driving assembly 16, two arms 36 disposed between the mounting plate 18 and the top plate 34. It should be noted that in this exemplary embodiment, base plate 22, mounting plate 18 and top plate 34 are all substantially horizontal and parallel to each other. Each of the two arms 36 includes a first load-measuring arm 38 and a second load-measuring arm 39 connected to the first load-measuring arm 38.

Specifically, in this exemplary embodiment, a bump 322 is provided on each diagonal position of the mounting plate 18, and one end of the first load-measuring arm 38 of the two arms 36 is respectively fixed to the two bumps 322, and the two first load-measuring arms 38 are parallel to each other and extend in opposite directions, so that the two first load-measuring arms 38 are horizontally suspended on the mounting plate 18 through the space. One end of the second load-measuring arm 39 is respectively connected to the other opposite end of the two first load-measuring arms 38 and extends parallel to each other in the opposite direction. The extension direction of the two second load-measuring arms 39 is the same as sliding rails 42, namely the back and forth direction of the intracavity surgical device such as catheter 32, extend in the same direction and are perpendicular to the extending directions of the two first load-measuring arms 38. The two second force-measuring arms 39 are positioned at two opposite sides of the intracavity surgical device such as catheter 32. That is, the two first load-measuring arms 38 and the two second load-measuring arms 39 are located on the four sides of, and sandwiched between, the mounting plate 18 and the top plate 34. The top plate 34 is installed on each respective opposite end of the two second load-measuring arms 39, so that the top plate 34 is horizontally suspended on the two second load-measuring arms 39 through the space. Driving assembly 16 is mounted on the top plate 34. In this exemplary embodiment, the two ends of the two first load-measuring arms 38 are fixedly attached to the diagonal positions of the mounting plate 18 relative to the four sides of the mounting plate 18, and the two ends of the two second load-measuring arms 39 are fixedly attached to the top plate 34. The diagonal position of 34 is relative to the four sides of top plate 34.

Therefore, the two first load-measuring arms 38 are configured to detect the linear force, and the two second load-measuring arms 39 are configured to detect the rotational torque. The operator (not shown) controls the movement of the conveyor assembly 20, via a controller (not shown), which subsequently drives the load measuring assembly 30 and the driving assembly 16 to move linearly toward or away from the subject. Resultantly, the driving assembly 16 drives the actuator 14 to move synchronously through a non-contact manner, so that the intracavity surgical device, such as catheter 32, clamped by the actuator 14 is delivered into or withdrawn from the surgical subject. The displacement of the intracavity surgical device 32 during delivery or withdrawal is obtained by the force measuring assembly 30 measuring displacement resistance and torque resistance when the device 32 is moved within the body of the patient.

Preferably, each first load-measuring arm 38 comprises a cantilever and patch sensors attached to two opposite vertical sides of the cantilever. The sensors are preferably the type of Strain Measurement Sensors (SMD) sensors. In this embodiment, one end of the two second load-measuring arms 39 is respectively connected to the other opposite end of the two first load-measuring arms 38 through a coupling block. Each second load-measuring arm 39 comprises a respective cantilever and patch sensors attached to two opposite horizontal sides of the cantilever. The heights of the placement of these sensors on the load-measuring arms are substantially the same. The load measuring assembly 30 should be such configured so that, when viewed in a top view of the robot assembly (the top view is not shown), the intracavity surgical device 32 should be parallel to and centered between the two second load-measuring arms 39.

It should be appreciated that alternatively, measuring each sensor component of the pair of two arms may also be achieved by measuring an array of sensors in a single unit of a cylindrical load cell. Variations of such are within the scope of the present disclosure.

Referring to FIG. 1-3, during the operation, when reciprocating conveyor 20 of the two catheter robot units are respectively at the initial positions, the two units of catheter operation assembly 10 are separated from each other. One may first let the actuator 14 of the catheter operation assembly 10 on the left in the figure to clamp the intracavity surgical device(catheter) 32, and the right actuator 14 of the catheter operation assembly 10 to loosen the catheter 32. At this moment, the yoke motor 24 of the left reciprocating conveyor 20 drives the scotch disk 26 to rotate causing piston linkage 28 to slide while sliding the half section up and down in sliding yoke 29. Being guided by rail slider 40, the load measuring assembly 30 and the driving assembly mounted on the slide block 44 cause driving assembly 16 to linearly move along the sliding rail 42, and synchronously drive the actuator 14 to move linearly towards the patient through the non-contact space control method. The left unit of the catheter operation assembly 10 gradually approaches the right unit of the catheter operation assembly 10 on the right side in FIG. 1, allowing the intracavitary surgical device 32 to be delivered into the patient. When the intracavity surgical device 32 is subjected to displacement resistance, it is transmitted to the load measuring assembly 30 through the left actuator 14 and the driving assembly 16, so that the displacement resistance is measured by the first load-measuring arm 38 of its two arms 36. Therefore, the adjustment on actuator 14 and driving assembly 16 can be made in real time.

When scotch disk 26 of the left scotch yoke 60 rotates half a circle to the maximum, that is, when the left catheter robot unit 200 is at the end position to its right, the left catheter robot unit 200 cannot continue to deliver the intracavity surgical device 32, this is the time to switch the operation to the right catheter robot unit 200. Alternatively, as the left catheter robot unit is about to release, the right catheter robot units configured to move with a matching the speed of the left and grab on to the surgical device so that the motion of the device is continuous as the left unit releases with the right unit preferably already be moving. At the moment, actuator 14 of the right-hand side clamps the intracavitary surgical device 32 such that actuator 14 of the left catheter robot unit 200 releases the intracavitary surgical device 32. At this moment, yoke motor 24 of the right reciprocating conveyor 20 drives the scotch disk 26 of scotch yoke 60 to rotate, sliding in and pushing sliding yoke 29 to drive the piston linkage 28 to slide halfway up and down in the sliding yoke 29. Subsequently, load measuring assembly 30 and the driving assembly 16 mounted on slide block 44 are induced to slide on the slide rail. Being guided by rail slider 40 and pushed by piston linkage 28, the load measuring assembly 30 and the driving assembly mounted on the slide block 44 move linearly along the sliding rail 42, and synchronously drive the actuator 14 to move linearly towards the patient through the non-contact space control method. The right catheter robot unit 200 gradually distances the left catheter robot unit 200 on the left side in FIG. 1, allowing the intracavitary surgical device 32 to be delivered into the patient. When the intracavity surgical device 32 is subjected to displacement resistance, it is transmitted to the load measuring assembly 30 through the right actuator 14 and the driving assembly 16. It is the difference (subtraction) of these two measurements in the pair of second load measuring arms 39 being used to measure the axial rotation torque. Therefore, the adjustment on actuator 14 and driving assembly 16 can be made in real time.

At this time, yoke motor 24 of the left reciprocating conveyor 20 continues to drive the scotch disk 26 to rotate the remaining half circle causing disk pin 27 slides up and down in the sliding yoke 29, pushing the piston linkage 28 for the remaining half. Similarly, load measuring assembly 30 and respective drive assembly 16 are driven linearly along the sliding rail 42 with the actuator 14 and gradually moves away from the right catheter robot unit 200. As a result, the left reciprocating conveyor 20 returns to the initial position. At the initial positions, the displacement resistance measured by the two first load-measuring arms 38 of the left load measuring assembly 30 is zero. In this embodiment, the sliding rail 42 of the left reciprocating conveyor 20 and the sliding rail 42 of the right reciprocating conveyor 20 are placed in the same straight line. Alternatively, the sliding rail 42 of the left reciprocating conveyor 20 and the sliding rail 42 of the right reciprocating conveyor 20 may be arranged parallel to each other.

When the scotch disk 26 of the right motion conversion mechanism 60 rotates half a circle at most, that is, when the right catheter robot unit 200 is at the end position, the right catheter robot unit 200 cannot continue to deliver the intracavity surgical device 32, as before, and then switch to the left delivery The actuator 14 of the module 10 clamps the intracavitary surgical device 32, and the actuator 14 of the right catheter robot unit 200 releases the intracavitary surgical device 32, and the yoke motor 24 of the left reciprocating conveyor 20 drives the rotating wheel of the motion conversion mechanism 60 again. Scotch disk 26 rotates, and the sliding yoke 29 of the scotch disk 26 slides half a section up and down again in the sliding yoke 29 of the piston linkage 28 and drives the piston linkage 28 to slide. 42 linearly moves, and the driving assembly 16 synchronously drives the operation assembly 14 to linearly move towards the patient through a non-contact space control method, and then approaches the catheter robot unit 200 on the right side again, allowing the intracavitary surgical device 32 to continue to be delivered into the patient. When the intracavity surgical device 32 is subjected to displacement resistance, it is transmitted to the load measuring assembly 30 through the left actuator 14 and the driving assembly 16, so that the displacement resistance is measured again by the first load-measuring arm 38 of its two arms 36 in real time. Same as before, at this moment, the yoke motor 24 of the reciprocating conveyor 20 on the right side continues to drive the scotch disk 26 of the motion conversion mechanism 60 to rotate for the remaining half circle, and the sliding column 68 of the scotch disk 26 slides up and down in the chute 66 of the piston linkage 28 for the remaining half, but the load measuring assembly 30 and the driving assembly 16 linearly move together with the actuator 14 along the sliding rail 42 and gradually approach the left catheter robot unit 200, so that the right reciprocating conveyor 20 returns to the initial position. The displacement resistance measured by the two first load-measuring arms 38 of the right force measuring assembly 30 is zero.

If the intracavity surgical device 32 needs to be withdrawn from the surgical subject, the operation in a reversal order can be performed. That is, in the above continuous delivery process, the order in which the left catheter robot unit 200 and the right catheter robot unit 200 clamping the intracavity surgical device 32 is reversed, in order to continuously withdraw the intracavity surgical device 32 from the subject. The order in which the displacement resistance is measured by the left load measuring assembly 30 and the right load measuring assembly 30 is also opposite to the foregoing.

For the non-contact spaced control method between the driving assembly 16 and the actuator 14, in addition to the above-mentioned driving assembly 16 driving the actuator 14 to move linearly synchronously, the driving assembly 16 can also control the respective actuator 14 to drive the intracavity medical equipment 32 to cause only a rotational movement or to move linearly and rotate simultaneously. When the intracavitary surgical device 32 is subjected to resistance when it rotates, it is also transmitted to the load measuring assembly 30 through the actuator 14 and the corresponding driving assembly 16 clamping the intracavity surgical device 32, so that the second load-measuring arm 39 of its two arms 36 measures the rotational torque resistance.

Whether it is moving or rotating, the force measuring assembly 30 can obtain the instantaneous resistance of the intracavity surgical device 32 in real time, which can be used for force and torque feedback. This not only enhances the operator's sense of presence in the operation allowing the operator to feel his/her way of conducting the operation, but also improves the accuracy of the operation. As a result, the safety and the efficiency of the operation are enhanced.

In addition, for the details of non-contact spaced control between the driving assembly 16 and the actuator 14 allowing the actuator 14 to drive the intracavity surgical device 32 to move linearly and/or rotate, one can refer to Chinese patent application 202210803401.5. Related contents of the surgical robot device and its operation method, including permanent magnet/electromagnetic drive, electromagnetic induction, electric field coupling, DC resonance, etc. are herein incorporated by reference. The “non-contact air-space control/transmission” refers to the control/transmission of the transmission mechanism without contact in space, rather than the control/transmission through the air medium, that is, it can also realize air-space control in a vacuum. That is to say, “non-contact air-space control/transmission” is driven by various field forces such as electric field force and magnetic field force without contact, and it can also be used in a vacuum. In a word, the use of the force of any field on the substance placed therein to realize “non-contact space control/transmission” is also applicable to the present application and within the scope of the present application.

It should also be understood that the variety of task of controlling as above mentioned, including the non-contact space control, the control of actuator 14, the control of the driving assembly 16, and the control of reciprocating conveyor may be conducted by a controller operated by a human operator, an AI operator or any combination of such.

In other embodiments, the load measuring assembly 30 may only include two first load-measuring arms 38 for measuring displacement resistance, or only include two second load-measuring arms 39 for measuring rotational torque resistance. At this time, the installation positions of the two first load-measuring arms 38 or the two second load-measuring arms 39 can be unchanged, but the two ends of the two first load-measuring arms 38 and the two second load-measuring arms 39 are pivotably fixed on the top plate 34. The two ends of the two second load-measuring arms 39 connected to the two first load-measuring arms 38 are pivotably fixed on the mounting plate 18.

In other embodiments, the two base plates 22 of the two transfer modules 20 can be combined into a single bottom plate, and the two slide rails 42 of the two slide rail slider mechanisms 40 can also be combined into a single slide rail.

The above-mentioned embodiments only express the limited implementation of the invention, and the description thereof is relatively specific and detailed, but should not be construed as limiting the scope of the present disclosure. It should be noted that, for those skilled in the art, some modifications, improvements or deteriorations can be made without departing from the inventive concept. The intracavity device delivery or pull and twist/rotation and real time force detection, of course, may include more than two catheter robot units 200 to complete the delivery or withdrawal of the intracavity surgical device 32, with real time force detection, the variations of such are within the scope of the present disclosure.

All or some of the steps in the procedures described above are listed sequentially, but in some cases the procedures may be performed in a different order than herein described.

The above-mentioned embodiments only describe some implementations of the present disclosure, and the descriptions thereof are relatively specific and detailed, but should not be construed as limiting the scope of the present disclosure. It should be appreciated that for those skilled in the art, without departing from the concept of the present disclosure, some modifications and improvements or deteriorations can also be made, such as whether the distal end of the guide wire is in front or not in the delivery process. The distal end of the catheter can be selected according to the actual situation, and these all belong to the protection scope of the present disclosure. Therefore, the protection scope of the patent for the present disclosure should be based on the appended claims.

It should be understood that the order of steps or order for performing certain action is immaterial so long as the described method remains operable. Moreover, two or more steps or actions may be conducted simultaneously.

What is claimed:

1. A robot apparatus comprising:
at least one robot unit configured to maneuver on an elongated surgical device;
a reciprocating conveyor having a motion converter converting rotary motions to linear motions to move the reciprocating conveyor; and
a controlling unit configured to control the motion converter and the at least one robot unit to cause the elongated surgical device to be inserted into or withdrawn from a surgical subject.

2. The robot apparatus of claim 1, wherein the motion converter is configured to convert rotary movement to linear movement.

3. The robot apparatus of claim 1, wherein the motion converter is a scotch yoke having a scotch disk, a disk pin fixed on an outer rim of the scotch disk, and a sliding yoke, the disk pin is configured to be fit into and slide along the sliding yoke.

4. The robot apparatus of claim 3, wherein the motion converter comprises a motor configured to drive the scotch disk to make rotary movements, causing the disk pin to push the sliding yoke to make reciprocating linear movements.

5. The robot apparatus of claim 4, wherein the reciprocating conveyor comprises a piston linkage, one side of the piston linkage is attached to the sliding yoke, the other side of the piston linkage is attached to the robot unit to cause the at least one robot unit to make the corresponding reciprocating linear movements.

6. The robot apparatus of claim 1, wherein the reciprocating conveyor further comprises a sliding rail onto which the at least one robot unit is mounted.

7. The robot apparatus of claim 1, wherein the at least one robot unit comprises a driving assembly and an actuator, the actuator is configured to have the elongated surgical device to be threaded through.

8. The robot apparatus of claim 7, wherein the actuator is configured to hold, release, or rotate the elongated surgical device.

9. The robot apparatus of claim 8, wherein the at least one robot unit include two robot units working with each other in such coordinated roles that when one of the two robot units is driven with the reciprocating linear movements while holding the elongated surgical device, the other of the two robot units is configured to release the elongated surgical device, followed by a next cycle in which the coordinated roles reverse between the two robot units to facilitate insertion or withdrawing of the elongated surgical device.

10. The robot apparatus of claim 7, wherein the actuator is mounted on the driving assembly and controlled by the driving assembly via a non-contact form of control.

11. The robot apparatus of claim 1 further comprises a base plate and wherein the reciprocating conveyor having a motor being mounted onto the base plate.

12. The robot apparatus of claim 10, wherein the reciprocating conveyor comprises a slider rail mounted onto the base plate.

13. The robot apparatus of claim 10 further comprises a mounting plate onto which the at least one robot unit is mounted, the mounting plate is also attached to the reciprocating conveyor.

14. The robot apparatus of claim 10 further comprises a mounting plate onto which a load measuring assembly is mounted, and a top plate mounted on top of the force measuring assembly.

15. The robot apparatus of claim 14, wherein the at least one robot unit is mounted on the top plate.

16. The robot apparatus of claim 14, wherein the load measuring assembly is installed between the top plate and the mounting plate and centered on the elongated surgical device.

17. The robot apparatus of claim 13, wherein the load measuring assembly comprises two pairs of arms, each pair of arms comprises a first load-measuring arm and a second force measuring arm.

18. The robot apparatus of claim 17, wherein the load measuring assembly further comprises a bump provided on each diagonal position of the mounting plate, one end of the first load-measuring arms respectively fixed to the respective bumps in the fashion of cantilevers.

19. The robot apparatus of claim 18, wherein the two first load-measuring arms of the two pairs of arms are parallel to each other and extend in opposite directions in such a way that the two first load-measuring arms are horizontally suspended on the mounting plate, and one end of the second load-measuring arm is respectively connected to the other opposite end of the respective first load-measuring arms, extending parallel to each other in the opposite direction.

20. The robot apparatus of claim 17, wherein the force measuring assembly comprises patch sensors of strain measurement attached to two opposite vertical sides of each of the two first load-measuring arms to detect linear force and attached to two opposite horizontal sides of each of the two second load-measuring arms to detect rotational torque.
